(19) United States
(12) Patent Application Publication
(10) Pub. No.: US 2005/0240121 A1
(43) Pub. Date: Oct. 27, 2005

(54) Method and Device for Providing of Pressure Dependent Biofeedback

(76) Inventors: Nadine Ferriss, Ottawa, ON (CA); Tammy Boucher, Ottawa, ON (CA)

Correspondence Address: D.F. Phenix & Associates;PATENT AND TRADE-MARK AGENTS, 135 Chemin Vigneaux, Cantley, QC, CA

(21) Appl. No.: 10/907,948

(22) Filed: Apr. 21, 2005

Related U.S. Application Data
(60) Provisional application No. 60/564,574, filed on Apr. 23, 2004.

(51) Int. Cl. A61B 5/103; A61B 5/117

(52) U.S. Cl. 600/587; 128/905

(57) ABSTRACT

A novel pressure dependent biofeedback device (PDBD) is disclosed. The PDBD is for being disposed in contact with an animate object, such as a portion of a human body of a user for having pressure exerted thereon by the user. The exerted pressure is measured and in response thereto, a biofeedback signal is generated. This biofeedback signal is then provided to the user in the form of biofeedback. The biofeedback is preferably visually based, however other forms are also envisaged such as sound and tactile.

This application claims priority from U.S. Provisional Application No. 60/564,574 filed Apr. 23, 2004.

### FIELD OF THE INVENTION

The field of the invention devices for use during physical exercise and more specifically to the field of devices that facilitate spinal alignment through a provision of biofeedback to a user.z

### BACKGROUND OF THE INVENTION

There is a growing need for core stabilization and strengthening among the current population. Individuals, such as those with low back pain, sacro-iliac joint dysfunctions, poor body or postural awareness and those who have undergone abdominal or low back surgery, typically have difficulty maintaining isometric contraction of their core muscles, such as the multifidus, transvers abdominus and pelvic floor muscles, while performing exercises. These people therefore typically require feedback to ensure correct performance of exercises.

For example, a fitness technique known as Pilates(™) is preferred for use in core strengthening: The major competitive strengths of Pilates(™) are core strengthening and increased motivation since it is generally done as a group exercise. However, none of the exercises are done in a standing position and classes can be fairy costly to attend. In addition, during the exercises, the individual has no indication as to whether or not proper spinal alignment is maintained and many spinal injuries are seen in physiotherapy from clients performing Pilates(™) exercises.

The Ab-Roller(™) has also been used for abdominal strengthening. However, studies have shown that compared to the standard abdominal crunch exercise, the Ab-Roller(™) showed no additional increase in use of the abdominal muscles during exercise, and no improvements in strength over a 18 week training period (Sept 1999, Demont R G, et al.). The Ab-Roller(™) is easy to use, supports the head which prevents neck pain and allows a smooth crunch. However, it is difficult to store due to its size. Also, arms may be used to contribute to the crunch. The Ab-Roller(™) does not incorporate transverse abdominus strengthening.

Another method that claims to increase muscle tone in various muscle groups is the use of electrical muscle stimulation. While studies show that electrical stimulation of a muscle over time will improve its ability to contract, there are no good studies that have found electrical stimulation to improve functional strength or human performance (May 2002, Porcari J P, et al.). Also, the electrical muscle stimulation will target the superficial abdominal muscles only and not transverse abdominus. Although the promise of no exercise and ease of use and storage appeals to the working market, it provides no improvement in functional strength. In addition, the self-adhesive pads tend to wear out thus requiring an ongoing purchase of components.

A technique used by physiotherapists to monitor spinal alignment during lower abdominal exercises is to place a hand under the patient's lumbar spine. The physiotherapist then provides feedback to the patient and thus increases the effectiveness of core strengthening. This technique has obvious drawbacks including the lack of independence during exercise.

The Pressure Cuff(™) is another device used that provides some feedback as to the pressure applied to it during exercise. It is used to monitor the position of the lumbar spine during the exercises and the feedback unit is connected to a pressure gauge. Air leakage, however, is a major problem with this device and it only provides visual feedback. Another drawback of the Pressure Cuff(™) is that it doesn't encourage symmetrical strengthening since it doesn't monitor both sides of the spine.

U.S. Pat. No. 6,019,738 also describes a device that provides feedback during exercise based on pressure applied to a sensing mechanism. The problem with this device, however, is that it is bulky to use, transport, and store. Furthermore, adjusting the unit to align with the curvature of the lumbar spine is done using inserts which may not accommodate all body types or user positions. Additionally, this device limits feedback that is provided to the user by using vibrator motors with optional auditory and visual signals. Therefore, there is a need for a device that teaches proper technique and body self awareness during exercise which focuses on the core muscles in addition to allowing various levels of difficulty to be achieved in order to maximize the development of core control. Furthermore, there is a need for a method and device that allows for convenient functional training in various positions without the problems of bulk, complexity and ineffectiveness.

It is therefore an object of the present invention to provide a device that teaches technique and body self awareness during exercise which focuses on the core muscles in addition to allowing various levels of difficulty to be achieved in order to maximize the development of core control. It is further an object of the present invention to provide a device that facilitates convenient functional training in various positions without the problems of bulk size, device complexity and device ineffectiveness. It is further an object of the present invention to provide a device that facilitates symmetrical strengthening of core muscles. Furthermore it is an object of the present invention to provide a device that is applicable for use with animate objects.

### SUMMARY OF THE INVENTION

In accordance with the invention there is provided a pressure dependent biofeedback device comprising: a pad having a first side and a second side opposite the first side; at least a fluid containing pocket disposed on the first side of the pad for adjustably conforming to a part of an animate object; a fluid varying device fluidly coupled with the at least a fluid containing pocket for varying a pressure of the fluid therein; at least a pressure sensor disposed in proximity of the at least a fluid containing pocket for measuring a pressure exerted thereon by being in contact with the part of an animate object and for generating at least a pressure signal; a control module coupled with the fluid varying device and the at least a pressure sensor for receiving the at least a pressure signal and for generating a biofeedback signal in dependence thereon; a biofeedback unit coupled with the control module for receiving of the biofeedback signal and for providing a visual representation thereof; and, a power source for providing of electrical power to the pressure dependent biofeedback device.

In accordance with the invention there is provided a pressure dependent biofeedback device comprising: a pad having a first side and a second side opposite the first side; at least an fluid containing pocket disposed on the first side of the pad for contacting a part of an animate object; at least a fluid varying device fluidly coupled with the at least a fluid containing pocket for varying a pressure of the fluid therein; at least a pressure sensor fluidly coupled with the at least a fluid containing pocket for measuring a pressure exerted thereon by a part of a human body and for generating at least a pressure signal; a control module coupled with the fluid varying device and the at least a pressure sensor for receiving the at least a pressure signal and for generating a biofeedback signal in dependence thereon; a biofeedback unit coupled with the control module for receiving of the biofeedback signal and for providing a visual representation thereof; and, a power source for providing of electrical power to the pressure dependent biofeedback device.

In accordance with the invention there is provided a method comprising: providing a pressure dependent biofeedback device comprising a biofeedback unit; disposing at least a portion of the pressure dependent biofeedback device in contact with a part of an animate object; applying pressure to the at least a portion of the pressure dependent biofeedback device; receiving of at least a pressure signal in dependence upon the applied pressure; determining a biofeedback signal in response to the applied pressure; and, providing of biofeedback in dependence upon the biofeedback signal.

### BRIEF DESCRIPTION OF THE DRAWINGS

Exemplary embodiments of the invention will now be described in conjunction with the following drawings, in which:

FIG. 1a illustrates a pressure dependent biofeedback device (PDBD) in accordance with a first embodiment of the invention;

FIG. 1b illustrates a pressure dependent biofeedback device (PDBD) in accordance with a second embodiment of the invention;

FIG. 1c illustrates a pressure dependent biofeedback device (PDBD) in accordance with a third embodiment of the invention, where the PDBD has an aperture formed therein for facilitating combined therapy;

FIG. 1d illustrates a pressure dependent biofeedback device (PDBD) in accordance with a fourth embodiment of the invention, where the PDBD has a wheel attached thereto for facilitating combined therapy and performance of advanced exercises;

FIGS. 1e and 1f illustrates a pressure dependent biofeedback device (PDBD) in accordance with a fifth embodiment of the invention, where the PDBD has at least a wheel attached thereto for rolling along a track for facilitating combined therapy and performance of advanced exercises;

FIGS. 1g and 1h illustrates a pressure dependent biofeedback device (PDBD) in accordance with a sixth embodiment of the invention for being attached to a backrest of a chair;

FIG. 1i illustrates a pressure dependent biofeedback device (PDBD) in accordance with a seventh embodiment of the invention, where the PDBD has first and second pockets disposed thereon for receiving of thermal packs for facilitating of combined therapy;

FIG. 1j illustrates a pressure dependent biofeedback device (PDBD) in accordance with a eighth embodiment of the invention, where the PDBD has first and second pockets disposed thereon for receiving of a electro stimulus pad for facilitating of combined therapy;

FIG. 2a illustrates one of the embodiments of the invention disposed underneath the back of a user from a side view, where in this position, the first and second fluid containing pockets are disposed on either side of the spine of the user thereby minimizing the pressure exerted on the spinal cord;

FIG. 2b illustrates a variation of one of the embodiments of the present invention where a sensitivity of the biofeedback that is provided to the user is optionally controllable by the user through adjustment of the sensitivity of the first and second pressure sensors by optionally providing of adjustable springs;

FIG. 3 illustrates a flow diagram for use of one of the embodiments of the invention, such as the embodiment shown in FIGS. 1a through 1j;

FIG. 4a illustrates a side view of operation of one of the embodiments of the invention, where the embodiment of the invention is positioned between a flat surface and a spine and the spine is in a neutral position; and,

FIG. 4b illustrates a side view of operation of one of the embodiments of the invention, where the embodiment of the invention is positioned between a flat surface and a spine with contraction of core muscles.

### DETAILED DESCRIPTION OF EMBODIMENTS OF THE INVENTION

Following are some definitions that facilitate the understanding of the embodiments of the invention. The term “isometric contraction” is used to define contraction in which a muscle exerts force but does not change in length. The term “neutral spine” is used to define a biomechanically correct spine position in which muscular balance is maintained. Dependent on the body of an individual a neutral lumbar spine, normal lumbar lordosis, may have to be determined by a worker skilled in the art.

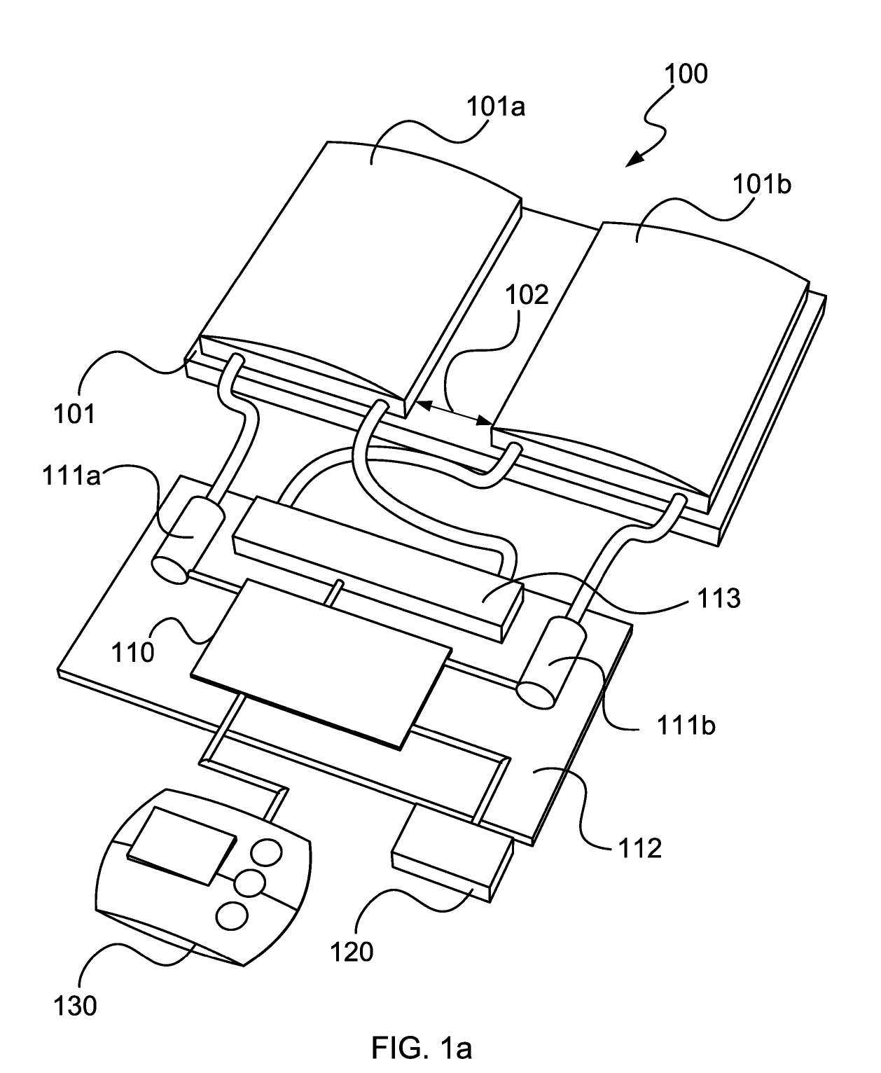

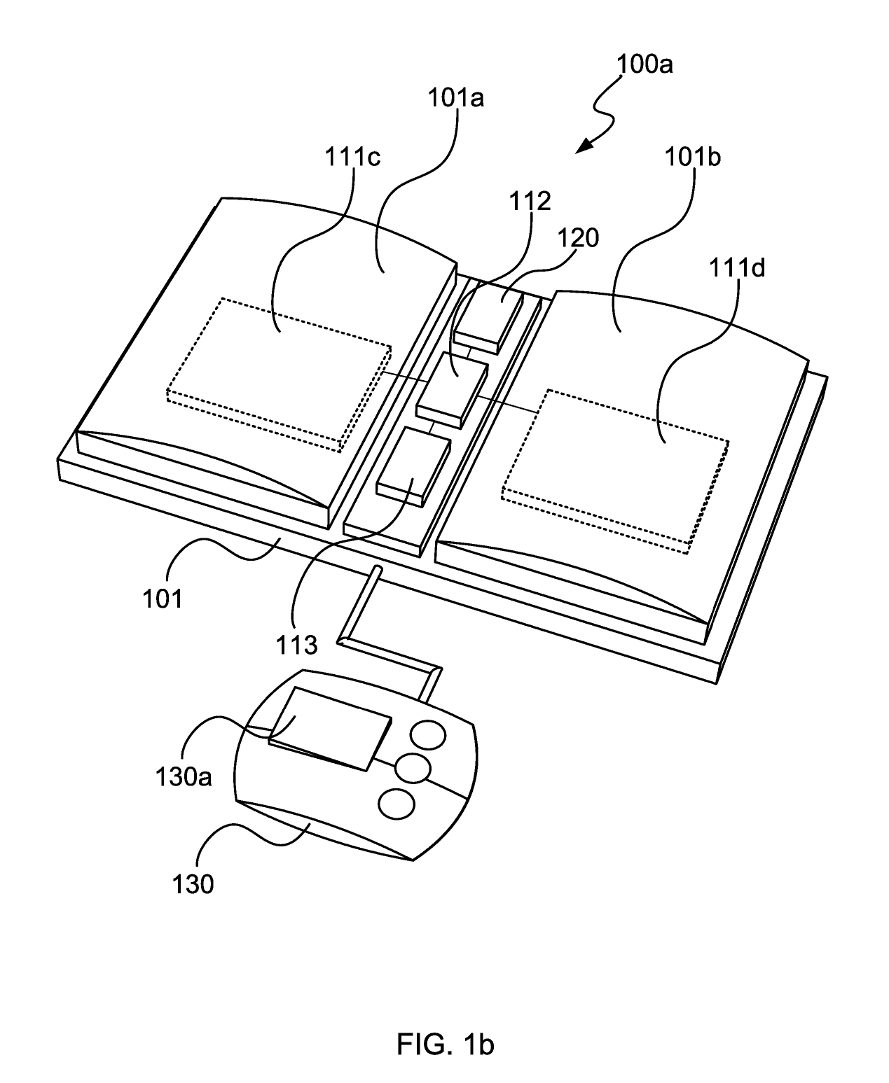

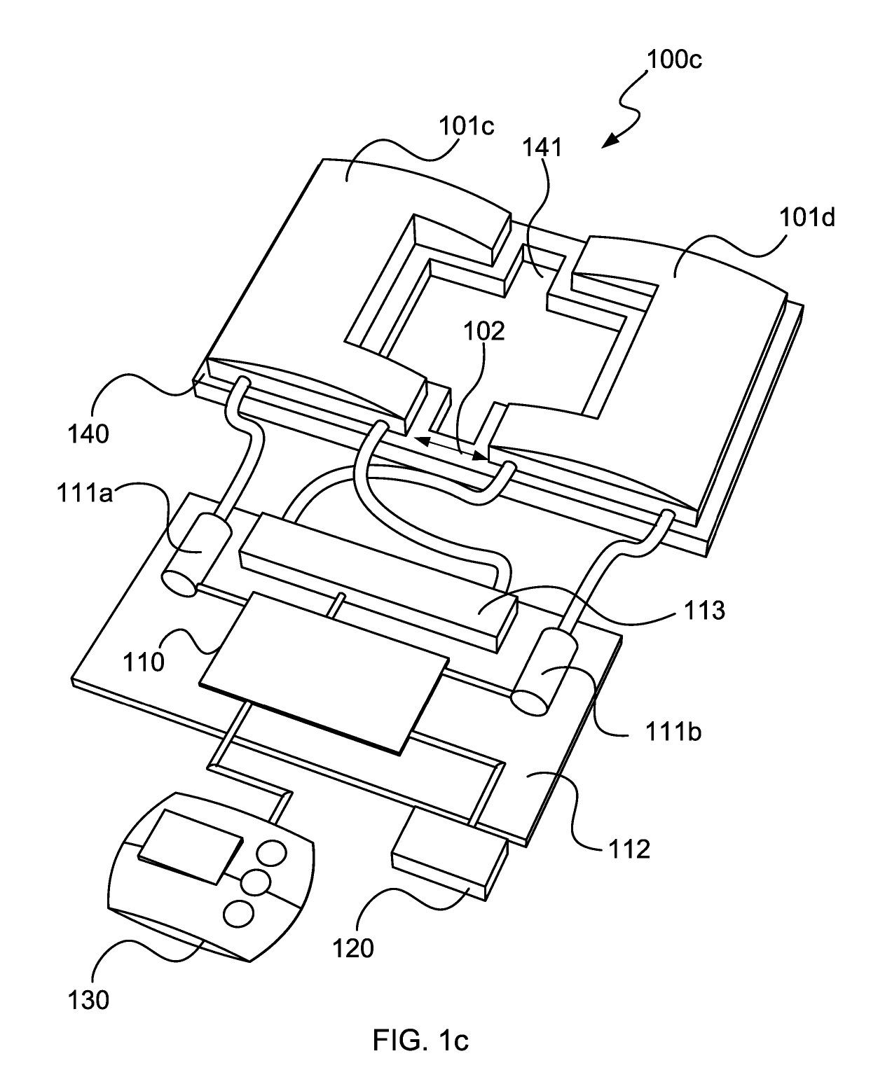

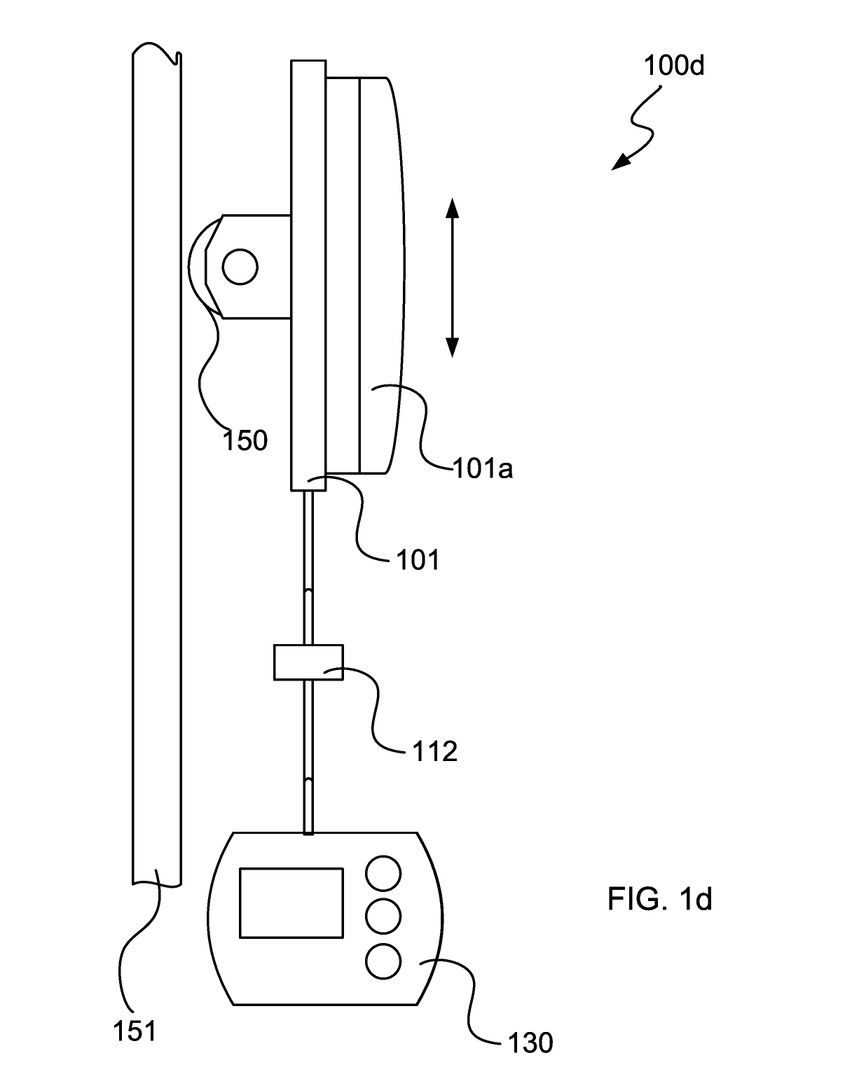

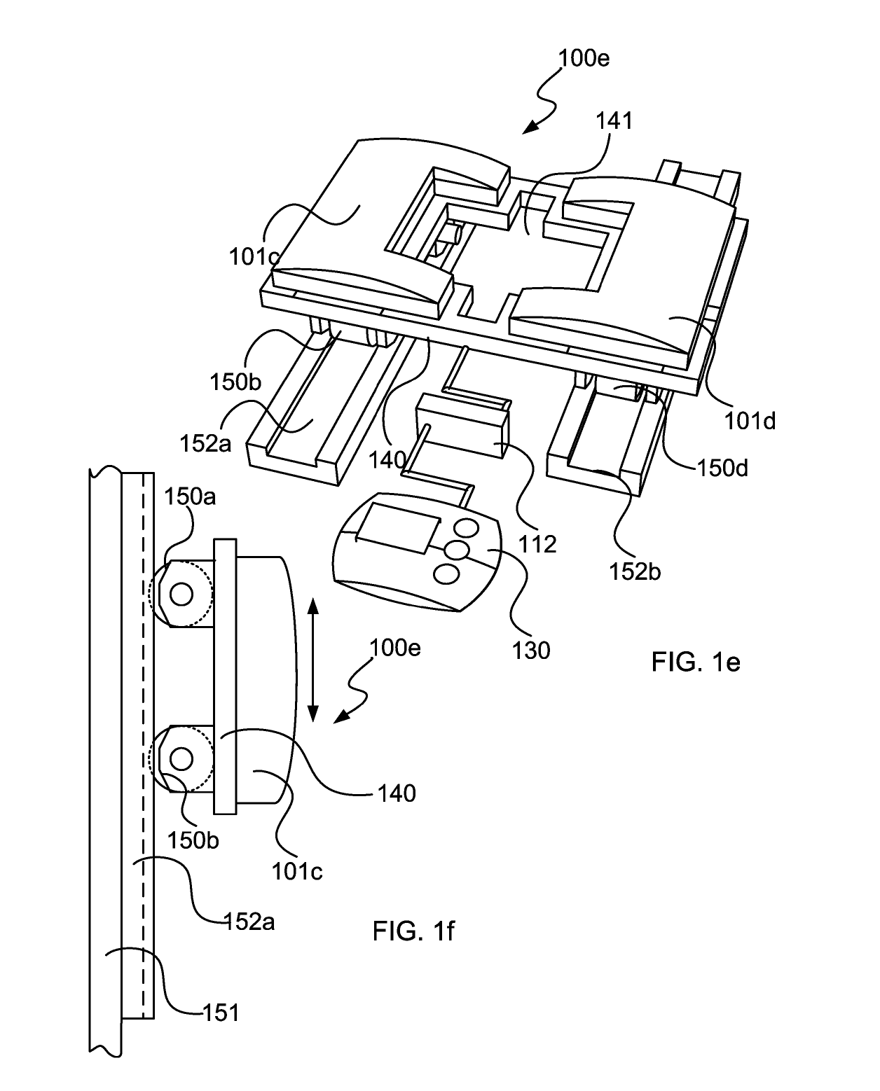

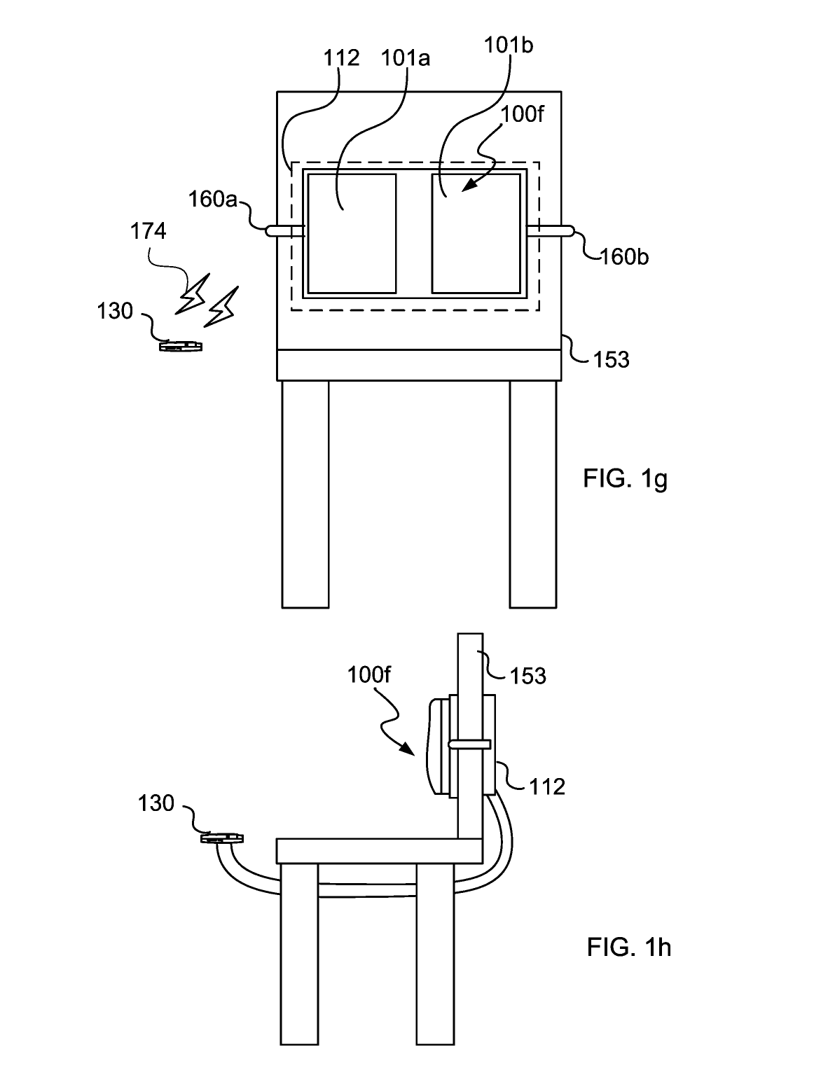

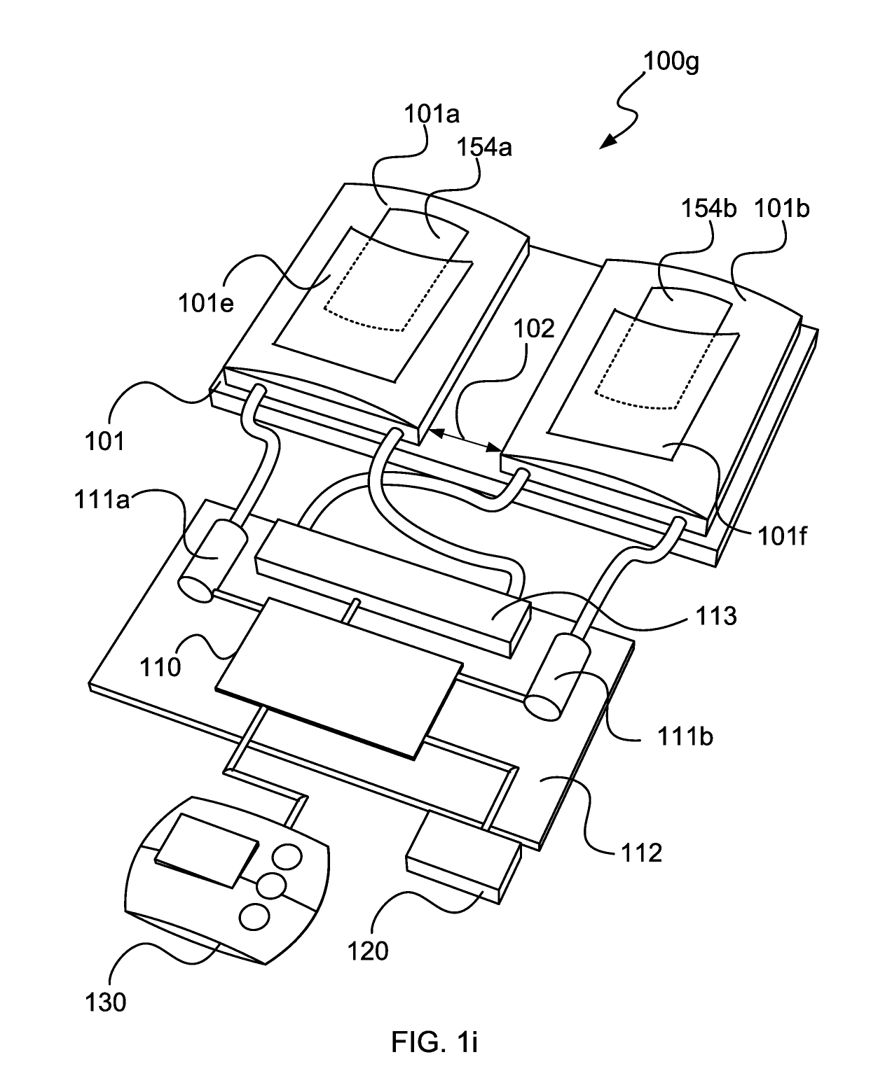

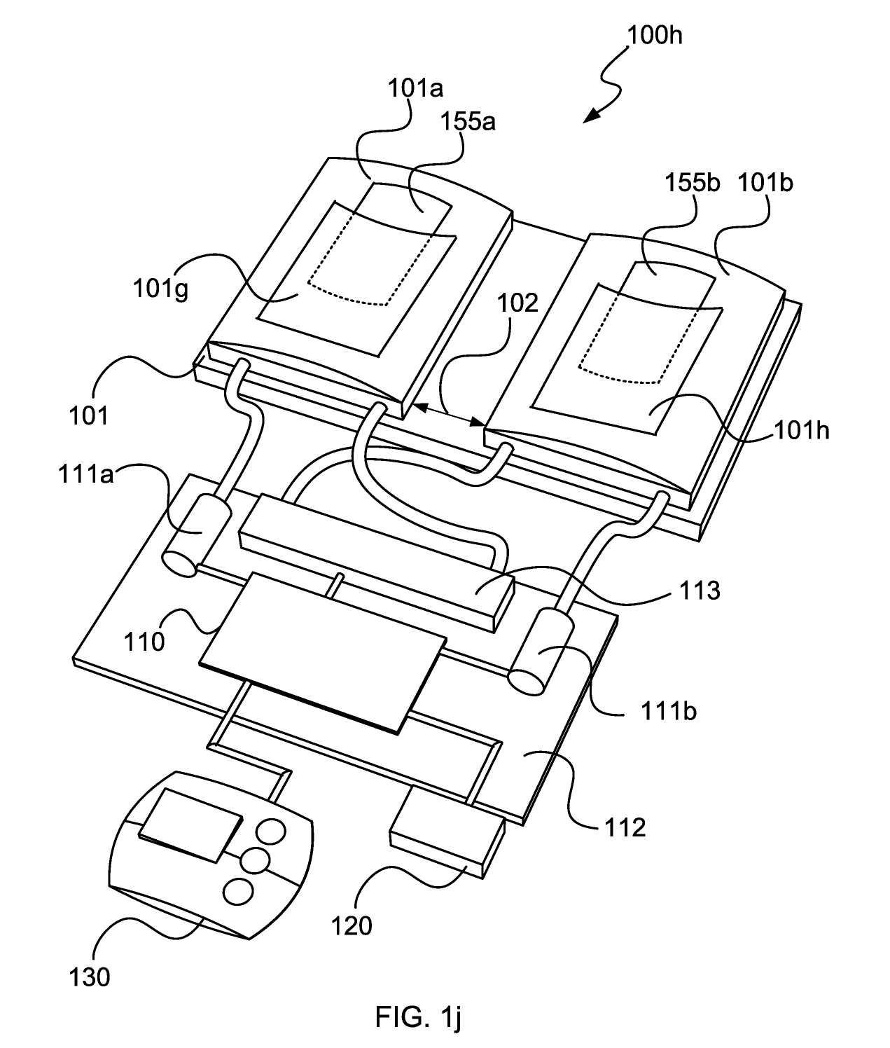

FIG. 1a illustrates a pressure dependent biofeedback device (PDBD) 100 in accordance with a first, and preferred, embodiment of the invention. The PDBD 100 comprises a pad 101 having a first fluid containing pocket 101a and a second fluid containing pocket 101b disposed on a first side of the pad 101. The first and second fluid containing pockets, 101a and 101b, are preferably spaced apart on the first side of the pad 101 by a distance 102.

A fluid varying device 113 is provided for varying the fluid level within the first and second fluid containing pockets, 101a and 101b. The fluid varying device is fluidly coupled with the first and second fluid containing pockets, 101a and 101b, for controllably varying fluid pressure thereof in order to vary at least a pressure of the fluid within the first and second fluid containing pockets, 101a and 101b. The fluid varying device 113 preferably comprises a fluid pump 113 that is fluidly coupled with the first and second fluid containing pockets, 101a and 101b, and at least a controllable valve for controllably varying of the fluid within the first and second fluid containing pockets. Optionally, a reservoir is fluidly coupled with the fluid varying device and the controllable valve in order to receive of excess fluid from the first and second fluid containing pockets, 101a and 101b.

A control module 112 is provided having a control circuit 110 preferably disposed therein. The control module 112 is electrically coupled with the first and second pressure sensors, 111a and 111b, for receiving of first and second pressure signals therefrom. The pressure sensors that are fluidly coupled with the first and second fluid containing pockets, 101a and 101b, using first and second tubes and are used to measure fluid pressure within these fluid containing pockets. As a response to the fluid pressure within the fluid containing pockets, the pressure sensors provide respective first and second pressure signals. Thus, as the fluid varying device varies fluid pressures within the first and second fluid containing pockets, 101a and 101b, the first and second pressure signals vary.

The fluid varying device 113 is preferably disposed within the control module 112 and is electrically coupled with the control circuit 110 for controllably varying fluid pressure within the first and second fluid containing pockets, 101a and 101b, in response to a control signal. A power source 120 is coupled with the control module 112 for providing of electricity thereto. The power source 120 either utilizes a plug type connector for receiving of AC current or is preferably in the form of a battery pack. The power source 120 is preferably disposed within the control module 112. However, it is also envisaged that at least part of the power source is disposed other than within the control module 112.

A biofeedback unit 130 is electrically coupled with the control module 112 for receiving of a biofeedback signal from the control circuit 110. The biofeedback unit provides biofeedback to the user in dependence upon the biofeedback signal, which is at least partially dependent upon the first and second pressure signals. Other parameters that affect the biofeedback signal are software programming within the control circuit 110. This software programming determines a relationship between the first and second pressure signals and the biofeedback that is to be provided to the user. The biofeedback that is provided to the user is preferably visually based or acoustically based, or a combination thereof, for providing visual or acoustic stimulation to the user.

Of course, other types of biofeedback such as mechanical based movements are also envisaged. In cases where the biofeedback is visually based, for example a LED display is utilized in the form of a bar graph in order to provide a varying number of bars when the pressure changes within at least one of the first and second inflatable tubes. Optionally, a LCD is utilized in order to convey information about the pressure changes within at least one of the first and second fluid containing pockets.

Other forms of the biofeedback signal are also contemplated, such as an auditory stimulus, a visual stimulus such as the LED indicator or an image, or a tactile stimulus such as a vibration. Of course, various combinations of these as biofeedback are also envisaged. Optionally of course the LED indicator is replaced with another type of visual indicator such as a LCD screen. Upon stimulation by the biofeedback, the user preferably varies the pressure they exert on the first and second fluid containing pockets, 101a and 101b, in order to facilitate therapy. As therapy varies in accordance with use of the PDBD 100 by the user, the biofeedback that the user receives is also varied. Through training and preferably with the aid of a practitioner in the art of core strengthening device therapy, the biofeedback that the user receives varies as the PDBD 100 is utilized in exercises. The further embodiments are discussed hereinbelow in many cases refer to the first embodiment.

It is further contemplated in the embodiments of the invention that the biofeedback signal is expressed as a soothing auditory biofeedback signal, such as soothing music or other sound effects, by the biofeedback unit 130. Optionally, a tactile stimulus such as a vibration is used to express the biofeedback signal to the user so that a massaging effect is provided to the user during use.

FIG. 1b illustrates a pressure dependent biofeedback device (PDBD) 100a in accordance with a second embodiment of the invention. The PDBD 100a comprises the pad 101 having the first fluid containing pocket 101a and the second fluid containing pocket 101b disposed on the first side of the pad 101. First and second fluid containing pockets, 101a and 101b, are preferably spaced apart on the first side of the pad 101 by the distance 102. The control module 112 is preferably disposed within the pad 101 and has first and second pressure sensors, 111c and 111d, which are respectively disposed of in proximity and preferably are contacting the first and second fluid containing pockets for detecting changes in pressure as a force is applied to at least one of the fluid containing pockets and for generating of first and second pressure signals. Disposed within the control module 112 is the control circuit 110 (not visible in this FIG) that is electrically coupled with the first and second pressure sensors, 111c and 111d, for receiving of the first and second pressure signals therefrom. Fluid varying device 113, is preferably disposed within the control module 112 and is coupled with the control circuit 110 for controllably varying fluid pressure of the first and second fluid containing pockets, 101a and 101b, which are fluidly coupled therewith. The power source 120 is coupled with the control module 112 for providing of electricity thereto. As in the first embodiment, the biofeedback unit 130 is electrically coupled with the control module 112 for receiving of the biofeedback signal from the control circuit 110 and for providing of biofeedback to the user in dependence upon the biofeedback signal.

FIG. 1c illustrates a pressure dependent biofeedback device (PDBD) 100c in accordance with a third embodiment of the invention. The PDBD 100c comprises a pad 140, similar to pad 101, having a first fluid containing pocket 101c and a second fluid containing pocket 101d disposed on the first side of the pad 140. In this case, the pad 140 comprises at least an aperture 141 formed therein. This aperture facilitates the use of acupuncture needles with electrical stimulus in conjunction with the PDBD 100c for providing of combined therapy to the user. The first and second fluid containing pockets, 101c and 101d, preferably other than have a same shape as the first and second fluid containing pockets, 101a and 101b.

FIG. 1d illustrates a pressure dependent biofeedback device (PDBD) 100d in accordance with a fourth embodiment of the invention. The PDBD 100d comprises the pad 101 having the first fluid containing pocket 101a and the second fluid containing pocket 101b (FIG. 1a, not visible in FIG. 1d) disposed on the first side of the pad 101. A wheel 150 is disposed on a second side of the pad 101, where the second side is opposite the first side having the first and second fluid containing pockets, 101a and 101b, disposed thereon. The wheel 150 is for contacting an approximately vertical surface 151 for facilitating moving of the PDBD 100d in an approximately vertical direction in a reciprocating manner. For example, this wheel 150 facilitates the use of the PDBD 100d while the user thereof is performing squat and heel lift exercises against the approximately vertical surface 151 for receiving of combined therapy.

FIG. 1e illustrates a front view of a pressure dependent biofeedback device (PDBD) 100e in accordance with a fourth embodiment of the invention. FIG. 1f illustrates the PDBD 100e from a side view. The PDBD 100e comprises the pad 140 having the first fluid containing pocket 101c and the second fluid containing pocket 101d (not visible in FIG. 1f) disposed on the first side of the pad 140.

A plurality of wheels 150a through 150d are disposed on the second side of the pad 101, similar to that shown in FIG. 1d, where the second side is opposite the first side has the first and second fluid containing pockets, 101c and 110d, disposed thereon. The plurality of wheels 150a through 150d are for contacting first and second tracks 152a and 152b (not visible in FIG. 1f) that are mounted to an approximately vertical surface 151 for facilitating moving of the PDBD 100e in an approximately vertical reciprocating manner where motion in an approximately vertical manner is approximately restricted because of the first and second tracks, 152a and 152b. Additionally, the pad 140 has at least the aperture 141 formed therein. This aperture facilitates using acupuncture needles with electrical stimulus in conjunction with therapy being administered while using the PDBD 100e. For example, the fourth embodiment of the invention facilitates using of the PDBD 100e while performing squat and heel lift exercises while simultaneously receiving the acupuncture needles with electrical stimulus. This provides combined therapy to the user. Optionally the PDBD 100e is not used with a vertical surface such as a chair, optionally the PDBD 100e is disposed on an approximately vertical surface, where the first and second tracks are disposed of in an approximately horizontal orientation.

FIG. 1g illustrates front view of a pressure dependent biofeedback device (PDBD) 100f, in accordance with a fifth embodiment of the invention, mounted to a backrest of a chair 153. FIG. 1h illustrates a side view of the PDBD 100f mounted to a backrest of the chair 153. The PDBD 100f comprises the pad 101 having the first fluid containing pocket 101a and the second fluid containing pocket 101b disposed on the first side thereof. In this embodiment, the control module 112 is disposed on a backside of the backrest of the chair 153, opposite the user, and first and second straps, 160a and 160b, couple the pad 101 and the first and second fluid containing pockets, 101a and 101b, to the control module 112. The biofeedback unit is coupled to the control module 112. Optionally, the control module 112 and the biofeedback unit 130 are formed as part of the same unit and are both held by the user for controllably adjusting of a fluid pressure within the first and second fluid containing pockets, 101a and 101b, while receiving of the biofeedback from the biofeedback unit 130. Referring to FIG. 1g, the biofeedback unit 130 is wirelessly coupled 174 to the control module 112, wherein as in FIG. 1h, the biofeedback unit 130 is coupled 174 to the control module 11 using a wire.

FIG. 1i illustrates a pressure dependent biofeedback device (PDBD) 100g in accordance with a sixth embodiment of the invention. The PDBD 100g comprises a pad 101 having a first fluid containing pocket 101a and a second fluid containing pocket 101b disposed on a first side of the pad 101. First and second pockets, 101e and 101f, are disposed on the first and second fluid containing pockets, 101a and 101b, for receiving of at least a thermal pack, in the form of first and second thermal packs, 154a and 154b. The first and second pockets, 101e and 101f are disposed opposite the first side of the pad 101 proximate the first and second fluid containing pockets, 101a and 101b, for contacting the user when using of the PDBD 100g. For example the first and second thermal packs, 154a and 154b, are either hot packs or cold packs for providing of heat or cold therapy to the user.

FIG. 1j illustrates a pressure dependent biofeedback device (PDBD) 100h in accordance with a sixth embodiment of the invention. The PDBD 100h comprises a pad 101 having a first fluid containing pocket 101a and a second fluid containing pocket 101b disposed on the first side of the pad 101. First and second pockets, 101g and 101h, are disposed on the first and second fluid containing pockets, 101a and 101b, for receiving a TENS electrical stimulus pads or an interferential current pad., 155a and 155b. The first and second pockets, 101g and 101h, are disposed opposite the first side of the pad 101 proximate the first and second fluid containing pockets, 101a and 101b, for contacting a user when using of the PDBD 100g. What is a general name for the TENS electro stimulating pads or the inferential current pad? This embodiment is for providing of combined therapy to the user.

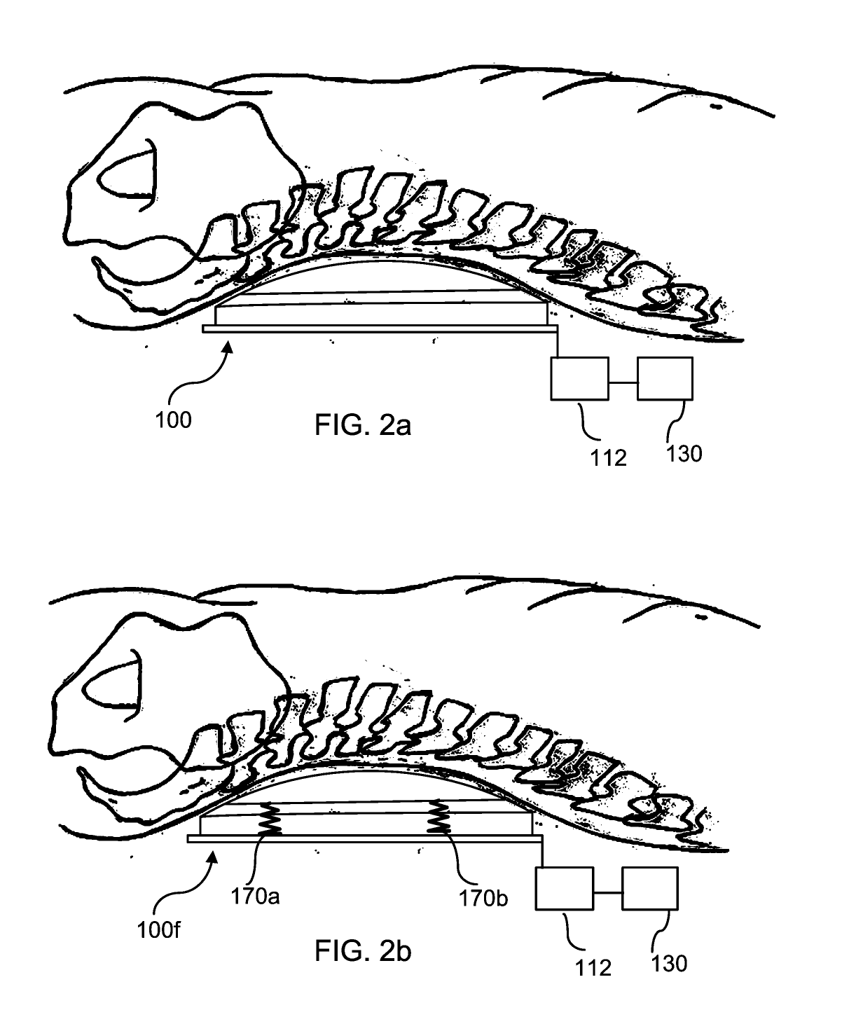

FIG. 2a illustrates one of the aforementioned embodiments of the invention, for example the first embodiment of the PDBD 100, but not limited to the first embodiment of the invention, disposed underneath the back of a user from a side view. In this position, the first and second fluid containing pockets are disposed on either side of the spine of the user thereby minimizing the pressure exerted on the spinal cord. The first and second fluid containing pockets are spaced apart by the distance 102 for accommodating the spine of the user between the fluid containing pockets, while allowing for the fluid containing pockets to conform to a lumbar curvature of the user maintained in neutral position. Because the lumbar curvature of the user is resting against the first and second fluid containing pockets this facilitates the monitoring of pressure exerted thereon by individual sides of the lumbar curvature of the spine. This monitoring of the pressure provides the first and second pressure signals to the control circuit for further determination of the biofeedback signal. Preferably the pad in accordance with the embodiments of the invention is manufactured from a thin foam type material or a plastic type material.

Preferably, the first and second fluid containing pockets are shaped such that they have a rectangular shape from a front view, however, various other shapes for the first and second fluid containing pockets are also envisaged to provide added levels of comfort to the user. Preferably the first and second fluid containing pockets are of such a shape to facilitate conforming to a curvature of a lower back of a user. For example, for those users with a smaller frame, first and second fluid containing pockets of a smaller size are envisaged. In addition, a plurality of first pockets and a plurality of second pockets is also envisaged for providing at least similar function as the first and second fluid containing pockets. Of course, different shapes for the pad in accordance with the embodiments of the invention are also envisaged.

Referring to FIG. 2b, a sensitivity of the biofeedback that is provided to the user is optionally controllable by the user through adjustment of the sensitivity of the first and second pressure sensors by optionally providing of a sensitivity adjustment mechanism, optionally in the form of adjustable springs, 170a and 170b, where adjusting of these adjustable springs varies the first and second pressure signals. Optionally, other spring mechanisms are also envisaged that utilize elastic or elastic foam type materials for providing a force against the lumbar curvature of the user. Preferably, the spring mechanism is adjustable such that the force exerted against the lumbar curvature of the user is variable. This variation of the force exerted by the spring mechanism is preferably varied in dependence upon a fitness of the user and their lumbar curvature. The sensitivity of the control circuit in response to the first and second pressure signals is also optionally varied in accordance with a predetermined functional relationship or by using of a lookup table, or through a combination thereof.

Optionally, during use of the PDBD in accordance with the aforementioned embodiments of the invention, first and second levels are set for at least one of the first and second pressure signals for providing of biofeedback to the user in response to the at least one of the first and second pressure signals. As a potential use of the aforementioned, the user sets min and max pressure values for the signals and thereafter the at least one of the first and second pressure sensors provides pressure variations between the min and max values as biofeedback to the user.

The PDBD in accordance with the aforementioned embodiments of the invention is potentially manufactured from various materials including neoprene, plastic or canvas, for example. In addition, the device is optionally self inflating and utilizes various types of valves that are fluidly coupled with the first and second fluid containing pockets, as well as with the fluid varying device 113 and the first and second pressure sensors for varying the fluid flow.

The fluid varying device is additionally useable for varying of fluids that are other than air, for example, liquids may be used, in which case the fluid varying device would pump the liquid in a controllable manner between the first and second fluid containing pockets in order to controllably provide inflation and deflation thereof. In this case, preferably a fluid reservoir is provided in order to contain excess fluid that is other than within the first and second fluid containing pockets. Optionally, a temperature varying device is coupled with the fluid in order to either cool or heat the fluid in dependence upon therapy that is to be received by the user. Preferably the temperature varying device is formed as part of the pad and disposed of in proximity of the first and second fluid containing pockets such temperature variations provided thereby are in proximity of a portion of the user. Of course, in other cases the temperature varying device is disposed within the control module.

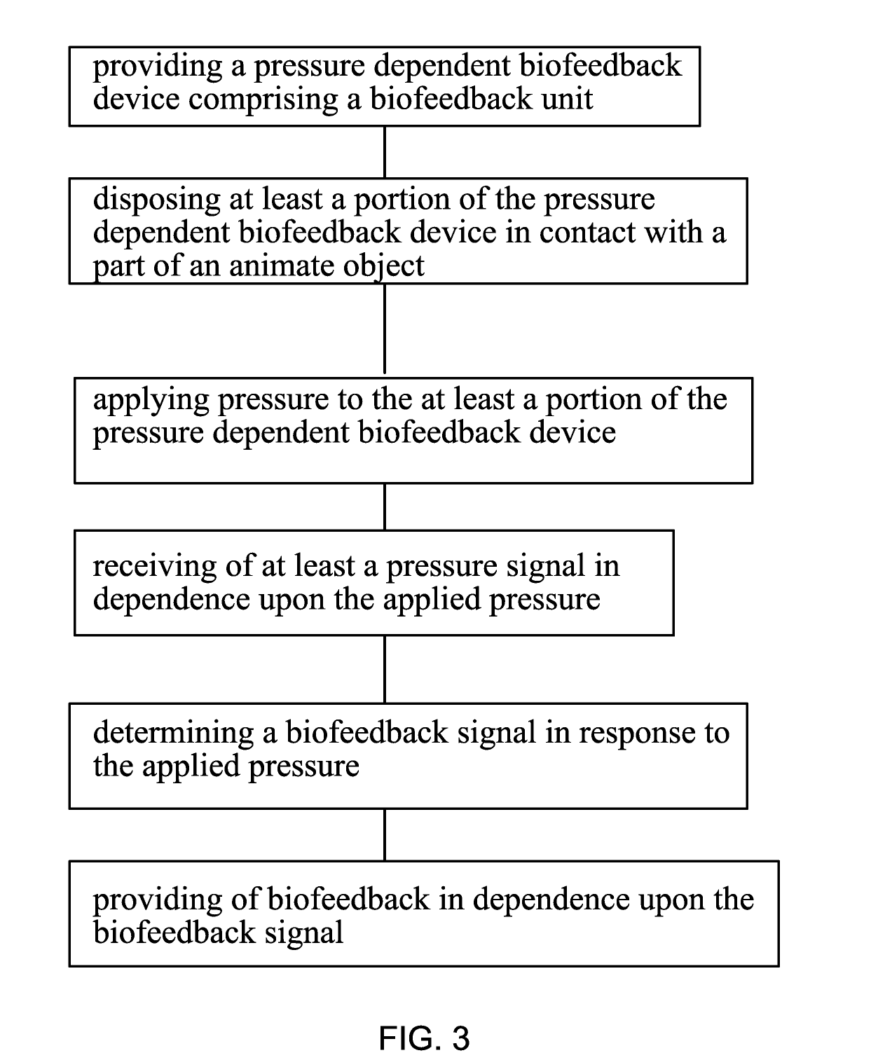

FIG. 3 illustrates a preferred method for using of the PDBD in accordance with the embodiments of the invention. Firstly, at least a portion of the PDBD is disposed in contact with a part of an animate object. The part of an animate object applies pressure to the PDBD. As the pressure signal is generated, it is provided to the control module for determination of a biofeedback signal in response to the applied pressure; and this is provided in the form of biofeedback. The animate object being either a body part of a human or a different type of animal.

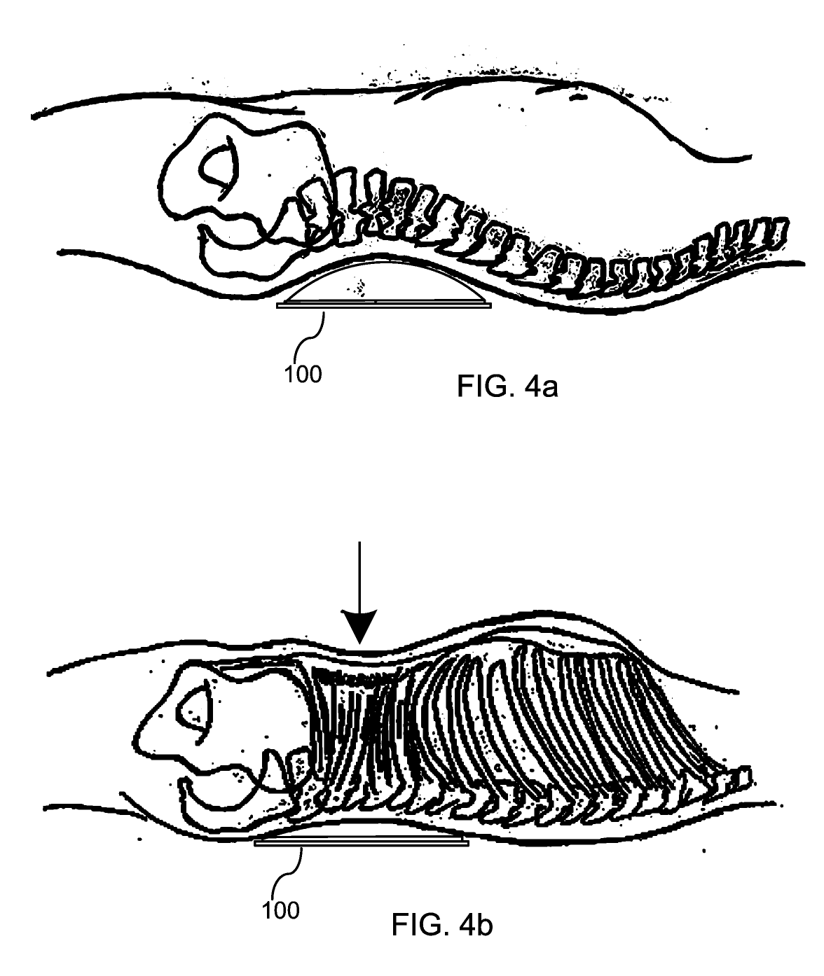

FIG. 4a illustrates one of the embodiments of the present invention, for example the first embodiment 100, positioned along the lower back of the user in the neutral position and ready for use. In accordance with the process flow in FIG. 3, the first and second fluid containing pockets 101a and 101b are inflated to conform to the user's lumbar curvature and the sensitivity of the control circuit in response to the first and second pressure signals is varied in dependence upon a the level of difficulty desired by the user. The control circuit 110 is powered on for providing of the biofeedback. As the user begins to flatten the lumbar spine by contraction of the transversus abdominus muscles, the first and second pressure signals and the biofeedback signal are generated. The biofeedback expresses to the user whether they are maintaining a preferable lower abdominal contraction in accordance with predetermined parameters. A preferable form of such a contraction is shown in FIG. 4b. Optionally, once the preferable form has been reached, the user continues to apply preferably the same pressure to the first and second fluid containing pockets 101a and 101b of the PDBD 100 while performing the exercises.

Advantageously, the embodiments of the PDBD in accordance with the present invention are also optionally used in combination with other physical training equipment. For example, the embodiments of the PDBD in accordance with the present invention are optionally used in conjunction with existing abdominal muscle training devices that aid in maximizing development of core muscle control. In addition, the embodiments of the PDBD in accordance with the present invention may be added to Nautilus (™) equipment or other exercise apparatus to ensure the user maintains proper spinal alignment during exercises, such as squats. A Sacro-Iliac belt is also optionally applied to the embodiments of the PDBD in accordance with the present invention to maintain the pelvis in proper alignment during dynamic and static muscle retraining of the pelvis girdle. Advantageously, when the user is finished with using of the embodiments of the PDBD in accordance with the present invention, the fluid containing pockets are deflated and thus facilitate transport and storage.

Although the above description has focused on exercises involving the core muscles of the body, it would be obvious to a worker skilled in the art that other devices for exercises involving other body parts of varying shapes and sizes, and various muscle groups, can be similarly designed to respond to first and second pressure signals to form the biofeedback signal. For example, a similar PDBD in accordance with the embodiments of the present invention is envisaged for use in strengthening the neck muscles.

Advantageously, the PDBD in accordance with the embodiments of the invention when used on conjunction with exercises known to those skilled in the art facilitates increasing strength of core muscles. Further advantageously, in accordance with the embodiments of the invention, symmetrical strengthening of core muscles by monitoring both sides of the lumbar spine is facilitated.

The embodiments of the present invention advantageously provide a method and apparatus for improving an individual's awareness of their spinal alignment during core stabilization and strengthening exercises by using of the biofeedback signal that is provided to the user. The embodiments of the invention thus facilitate core strengthening by encouraging a spinal position suitable for core stabilization and core strengthening exercises.

Numerous other embodiments may be envisaged without departing from the spirit or scope of the invention.

1. A pressure dependent biofeedback device comprising:
a pad having a first side and a second side opposite the first side;
at least a fluid containing pocket disposed on the first side of the pad for adjustably conforming to a part of an animate object
a fluid varying device fluidly coupled with the at least a fluid containing pocket for varying a pressure of the fluid therein;
at least a pressure sensor disposed in proximity of the at least a fluid containing pocket for measuring a pressure exerted thereon by being in contact with the part of an animate object and for generating at least a pressure signal;
a control module coupled with the fluid varying device and the at least a pressure sensor for receiving the at least a pressure signal and for generating a biofeedback signal in dependence thereon;
a biofeedback unit coupled with the control module for receiving of the biofeedback signal and for providing a visual representation thereof; and,
a power source for providing of electrical power to the pressure dependent biofeedback device.

2. A pressure dependent biofeedback device according to claim 1 comprising:
an aperture formed within the pad approximately adjacent the at least a fluid containing pocket for facilitating administration of combined therapy to the user.

3. A pressure dependent biofeedback device according to claim 1 comprising:
at least a wheel disposed on the second side of the pad for contacting a surface for facilitating motion of the pressure dependent biofeedback device along the surface.

4. A pressure dependent biofeedback device according to claim 3 comprising:
at least a track disposed along the surface for receiving of the at least a wheel for facilitating motion of the pressure dependent biofeedback device along at least one track.

5. A pressure dependent biofeedback device according to claim 1 wherein the at least a fluid containing pocket comprises a first fluid containing pocket and a second fluid containing pocket, where there is a separation between the first and second fluid containing pockets for accommodating a spine of the user.

6. A pressure dependent biofeedback device according to claim 5 comprising:
an aperture formed within the pad in proximity to the separation formed between the first and second fluid containing pockets for facilitating administration of combined therapy to the spine of the user.

7. A pressure dependent biofeedback device according to claim 1 wherein the at least a fluid containing pocket, the fluid varying device, the at least a pressure sensor and the control module are formed within the pad.

8. A pressure dependent biofeedback device according to claim 1 comprising a first strap and a second strap disposed between the pad and the control module for disposing the pad on a backrest of a chair and disposing the control module on a side of the chair opposite the backrest, wherein the first and second straps couple the pad and the control module for frictionally engaging the chair therebetween.

9. A pressure dependent biofeedback device according to claim 1 comprising at least a pocket disposed on a surface of the at least a fluid containing pocket opposite the first side of the pad, wherein the pocket is for receiving of at least one of a thermal pack and an electrical stimulus pad.

10. A pressure dependent biofeedback device according to claim 1 wherein the fluid is at least one of air and liquid.

11. A pressure dependent biofeedback device according to claim 1 comprising a heater for heating the fluid.

12. A pressure dependent biofeedback device according to claim 1 comprising a sensitivity adjustment mechanism for adjusting a response of the at least a pressure signal to pressure exerted on the at least an inflatable pocket by a part of the human body.

13. A pressure dependent biofeedback device according to claim 1 comprising a sacroiliac belt for facilitating maintaining of spinal alignment during use of the pressure dependent biofeedback device.

14. A pressure dependent biofeedback device according to claim 1 wherein the coupling is at least one of a wired coupling and a wireless coupling.

15. A pressure dependent biofeedback device according to claim 1 wherein the fluid varying device comprises at least one of a fluid pump that is fluidly coupled with the at least a fluid containing pocket and at least a controllable valve for controllably varying of the fluid within the at least a fluid containing pocket.

16. A pressure dependent biofeedback device according to claim 1 wherein the animate object is a human having the pressure dependent biofeedback device conformed to a lumbar curvature of the back.

17. A pressure dependent biofeedback device comprising:
a pad having a first side and a second side opposite the first side;
at least an fluid containing pocket disposed on the first side of the pad for contacting a part of an animate object;
at least a fluid varying device fluidly coupled with the at least a fluid containing pocket for varying a pressure of the fluid therein;
at least a pressure sensor fluidly coupled with the at least a fluid containing pocket for measuring a pressure exerted thereon by a part of a human body and for generating at least a pressure signal;
a control module coupled with the fluid varying device and the at least a pressure sensor for receiving the at least a pressure signal and for generating a biofeedback signal in dependence thereon;
a biofeedback unit coupled with the control module for receiving of the biofeedback signal and for providing a visual representation thereof; and,
a power source for providing of electrical power to the pressure dependent biofeedback device.

18. A pressure dependent biofeedback device according to claim 10 comprising:
at least a wheel disposed on the second side of the pad for contacting a surface for facilitating motion of the pressure dependent biofeedback device along the surface.

19. A pressure dependent biofeedback device according to claim 18 comprising:
at least a track disposed along the surface for receiving of the at least a wheel for facilitating motion of the pressure dependent biofeedback device along the at least a track.

20. A pressure dependent biofeedback device according to claim 19 comprising:
an aperture formed within the pad for facilitating administration of combined therapy to the user.

21. A pressure dependent biofeedback device according to claim 10 wherein the at least a fluid containing pocket comprises a first fluid containing pocket and a second fluid containing pocket, where there is a separation between the first and second fluid containing pockets for accommodating a spine of the user.

22. A pressure dependent biofeedback device according to claim 21 comprising:
an aperture formed within the pad in proximity to the separation formed between the first and second fluid containing pockets for facilitating administration of combined therapy to the spine of the user.

23. A pressure dependent biofeedback device according to claim 10 wherein the at least a fluid containing pocket, the fluid varying device, the at least a pressure sensor and the control module are formed within the pad.

24. A pressure dependent biofeedback device according to claim 10 comprising at first strap and a second strap disposed between the pad and the control module for disposing the pad on a backrest of a chair and disposing the control module on a side of the chair opposite the backrest, wherein the first and second straps couple the pad and the control module for frictionally engaging the chair therebetween.

25. A pressure dependent biofeedback device according to claim 10 comprising at least a pocket disposed on a surface of the at least a fluid containing pocket opposite the first side of the pad, wherein the pocket is for receiving of at least one of an electrical stimulus pad and a thermal pack.

26. A pressure dependent biofeedback device according to claim 10 wherein the fluid is at least one of air and liquid.

27. A pressure dependent biofeedback device according to claim 26 comprising a heater for heating the fluid.

28. A pressure dependent biofeedback device according to claim 10 comprising a sensitivity adjustment mechanism for adjusting a response of the at least a pressure signal to pressure exerted on the at least an inflatable pocket by a part of the human body.

29. A pressure dependent biofeedback device according to claim 10 comprising a sacroiliac belt for facilitating maintaining of spinal alignment during use of the pressure dependent biofeedback device.

30. A pressure dependent biofeedback device according to claim 10 wherein the coupling is at least one of a wired coupling and a wireless coupling.

31. A method comprising:
providing a pressure dependent biofeedback device comprising a biofeedback unit;
disposing at least a portion of the pressure dependent biofeedback device in contact with a part of an animate object;
applying pressure to the at least a portion of the pressure dependent biofeedback device;
receiving of at least a pressure signal in dependence upon the applied pressure;
determining a biofeedback signal in response to the applied pressure; and,
providing of biofeedback in dependence upon the biofeedback signal.

32. A method according to claim 31 comprising adjusting of a sensitivity of the biofeedback signal in response to the applied pressure.

33. A method according to claim 31 comprising varying a response of the biofeedback signal in response to the applied pressure in a predetermined manner.

34. A method according to claim 31 comprising providing a lookup table having at least a predetermined relationship stored therein between the at least a pressure signal and the biofeedback signal.

35. A method according to claim 31 wherein the biofeedback signal is at least one of triggered by the part of an animate object exerting pressure on the at least a fluid containing pocket and is derived from pressure in response to the part of an animate object exerting pressure on the at least a fluid containing pocket.

36. A method according to claim 31 comprising:
providing of first and second levels that are predetermined for at least one of the first and second pressure signals; and,
providing of biofeedback in response to the at least one of the first and second pressure signals in relation to the first and second levels thereof.

37. A method according to claim 31 wherein the at least a part of the animate object is a portion of one of a human body and an animal body.
